(19) United States
(12) Patent Application Publication
(10) Pub. No.: US 2017/0367322 A1
(43) Pub. Date: Dec. 28, 2017

(54) Reconstitution Solution For Spray-Dried Plasma

(71) Applicant: Velico Medical, Inc., Beverly, MA (US)

(72) Inventors: Qiyong Peter Liu, Newton, MA (US); Ryan Christopher Carney, Hudson, NH (US)

(73) Assignee: Velico Medical, Inc.

(21) Appl. No.: 15/481,692

(22) Filed: Apr. 7, 2017

Related U.S. Application Data
(60) Provisional application No. 62/319,651, filed on Apr. 7, 2016.

(51) Int. Cl. A01N 1/02; A61K 35/16; G01N 33/86

(52) U.S. Cl. CPC A01N 1/0205; G01N 33/86; A01N 1/0278; A61K 35/16

(57) ABSTRACT

The present invention relates to a reconstitution solution for spray dried plasma having a non-anticoagulant compound that does not bind calcium. When the reconstitution solution of the present invention is mixed with spray dry plasma, the reconstituted plasma mediates platelet adhesion and aggregation about the same as or greater than the starting plasma prior to spray drying. The present invention also relates to an assay for determining platelet adhesion and aggregation using microfluidic flow cell system having a shear flow. The assay assesses labeled whole blood samples having reconstituted plasma having spray dried plasma and a reconstitution solution; platelets; and red blood cells. After inducing a shear flow under conditions suitable for clot formation, coverage area of the platelets, intensity of the platelets, morphology, or a combination thereof is detected to determine platelet accumulation (e.g., platelet adhesion and aggregation).

### RELATED APPLICATION

This application claims the benefit of U.S. Provisional Application No. 62/319,651, entitled, “Reconstitution Solution For Spray-Dried Plasma” by Qiyong Peter Liu et al., filed Apr. 7, 2016.

### GOVERNMENT SUPPORT

The invention was supported, in whole or in part, by a grant HHS0100201200005C from the Biomedical Advanced Research and Development Authority (BARDA). The Government has certain rights in the invention.

The entire teachings of the above applications are incorporated herein by reference.

### BACKGROUND OF THE INVENTION

Making up about 55% of the total volume of whole blood, blood plasma is a whole blood component in which blood cells and other constituents of whole blood are suspended. Blood plasma further contains a mixture of over about 700 proteins and additional substances that perform functions necessary for bodily health, including clotting, protein storage, and electrolytic balance, amongst others. When extracted from whole blood, blood plasma may be employed to replace bodily fluids, antibodies and clotting factors. Accordingly, blood plasma is often used in medical treatments.

To facilitate storage and transportation of blood plasma until use, plasma is typically preserved by freezing soon after its collection from a donor. Fresh-Frozen Plasma (FFP) is obtained through a series of steps involving centrifugation of whole blood to separate plasma and then freezing the collected plasma within less than 8 hours of collecting the whole blood. In the United States, the American Association of Blood Banks (AABB) standard for storing FFP is up to 12 months from collection when stored at a temperature of −18° C. or below. FFP may also be stored for up to 7 years from collection if maintained at a temperature of −65° C. or below. In Europe, FFP has a shelf life of only 3 months if stored at temperatures between −18° C. to −25° C., and for up to 36 months if stored at colder than −25° C. If thawed, European standards dictate that the plasma must be transfused immediately or stored at 1° C. to 6° C. and transfused within 24 hours. If stored longer than 24 hours, the plasma must be relabeled for other uses or discarded.

Notably, however, FFP must be kept in a temperature-controlled environment of −18° C. or colder throughout its duration of storage to prevent degradation of certain plasma proteins and maintain its efficacy, which adds to the cost and difficulty of storage and transport. Furthermore, FFP must be thawed prior to use, resulting in a delay of 30-80 minutes before it may be used after removal from cold storage.

An alternative to FFP is spray dried plasma. Spray dried plasma does not need to be continuously stored in an environment of −18° C. or colder and therefore had an advantage over FFP. Generally, spray drying plasma involves aerosolizing the plasma droplets and drying them so that a spray dried power is formed. Certain spray drying techniques are being developed and optimized to obtain effective and functional plasma to be transfused into patients.

Accordingly, there is a need to improve spray drying techniques that provide plasma, once transfused into patients, that allows for effective clotting. There is a further need to optimize spray drying techniques so that the plasma that is reconstituted is a good alternative to FFP.

### SUMMARY OF THE INVENTION

The inventors have optimized the spray drying plasma process and have made a number of discoveries pertaining to the present invention which include a novel reconstitution solution to be used to reconstitute spray dried plasma. The novel reconstitution solution of the present invention includes a compound that is referred to as a Non-AntiCoagulant that does Not bind Calcium (NACNC) and results in a reconstituted spray dried plasma that performs similar to, and in certain cases better than the starting plasma, e.g., Fresh Frozen Plasma (FFP). The inventors discovered, contrary to accepted dogma, that vWF multimers appear not to need to be high molecular weight multimers in order to be effective in clotting (e.g., in platelet adhesion and aggregation). In particular, they determined that intermediate or low molecular weight vWF multimers are effective as long as the reconstitution solution does not include a component that binds calcium. Another discovery relates to the use of a NACNC compound. It was discovered that a NACNC compound, such as glycine HCl, can be used successfully as a buffer for reconstituting dried plasma such as spray dried plasma (SpDP) while not binding calcium during bleeding events involving platelet adhesion and aggregation. It was further determined that spray dried plasma reconstituted with a NACNC compound such as glycine HCl is similar or superior to FFP in controlling a bleeding environment as determined by testing platelet adhesion and aggregation in vitro by flow cell assays such as that performed on the BIOFLUX 1000 system (Fluxion Biosciences, Inc.). Yet another discovery relates to the present invention is that a cell flow assay can be employed in a novel way to provide an accurate and improved in vitro model for bleeding control to test a spray dried plasma formulations.

In particular, the present invention relates to a reconstitution solution for use in reconstituting spray dried plasma, wherein the reconstitution solution includes at least one (e.g., one or more) NACNC in the range between about 5 mM and about 20 mM (e.g., about 8 and about 16 mM) total concentration and water. When two or more NACNC compounds are used, in an embodiment, the total concentration of the combined NACNC compounds ranges between about 5 mM and about 20 mM. In an embodiment, the reconstitution solution can have NACNC compounds and additional compounds believed to improve the solution and use of plasma. Examples of NACNC include glycine HCl, ascorbic acid, lactic acid, gluconic acid and any combination thereof. Similarly, in an embodiment, additional candidates for use as a NACNC in the reconstitution solution of the present invention can be assessed for its ability to interfere with coagulation assays and/or binds to calcium by measuring aPTT or R-time of TEG. aPTT measures the activity of the intrinsic and common pathways of coagulation and the R-time of TEG refers to the rate at which an initial clot formation is detected. The aPTT test is performed on an Instrumentation Laboratory ACL Top coagulation analyzer. R-time is a reflection of the coagulation factor cascade (thrombin generation and fibrin formation) and is tested by Thrombelastograph Hemostasis Analyzer. In the case of testing using aPTT and R-time, the NACNC candidate should not have prolonged aPTT and R-times, as compared to a positive control, such as glycine HCl, a compound that has proven to be a good reconstitution solution compound, allowing reconstituted plasma to be effective in clot formation (e.g., platelet aggregation and adhesion).

In an embodiment, when spray dried plasma is reconstituted using the reconstitution solution of the present invention, the reconstituted plasma (once combined with platelets) works about as well as starting plasma, for example, with respect to clot formation and its clotting properties. In an embodiment, starting plasma is plasma that is not frozen (e.g., never-frozen plasma) or thawed FFP. Clotting properties of the reconstituted plasma can be measured by using methods known in the art, and include, in an embodiment, platelet adhesion/aggregation (e.g., using a microfluidic flow cell system that induces a shear flow). Throughout the application, reference to platelet adhesion/aggregation function and performance is made with respect to reconstituted plasma, and such a reference, when appropriate, refers to the reconstituted plasma after combination with at least platelets and preferably red blood cells. In an aspect, other methods of assessment, now known or developed in the future, can be used to determine the clotting properties of the reconstituted plasma.

In an embodiment, the platelet adhesion, aggregation or both of the reconstituted plasma, once combined with platelets and preferably red blood cells, of the present invention are about the same as or greater than, the starting plasma. Various methods can be used to measure platelet adhesion, aggregation or both. In an embodiment, platelet adhesion and/or platelet aggregation can be measured using a flow cell assay by testing the whole blood reconstituted from platelets, red cells, and a sample (rehydrated spray dried plasma (SpDP), or FFP) alone, or in combination with a volume of normal plasma that does not have an anticoagulant with calcium chelators such as citrate or EDTA, under arterial shear, pathological shear, or both. In an embodiment, under the arterial shear or pathological shear, or both, the rate of platelet accumulation (e.g., platelet aggregation and adhesion) of the reconstituted plasma of the present invention is at least about 1% to about 4× greater, as compared to rate of platelet accumulation (e.g., platelet aggregation) of the starting plasma (e.g., FFP).

The present invention also relates to methods for reconstituting spray dried plasma by combining the reconstitution solution, described herein, with spray dried plasma, to obtain reconstituted plasma. In an aspect, the method further includes mixing or shaking the reconstituted plasma to obtain a uniform mixture. Such reconstitution can occur in a plasma bag or container.

Accordingly, the present invention also pertains to a plasma bag or container having a first reconstitution container for storing the reconstitution solution, as described herein, a second plasma container for storing spray dry plasma; and a connector that communicates between the first container and the second container, the connector having a barrier that can be broken (e.g., a frangible barrier) to allow the reconstitution solution to mix with the plasma in the second plasma container.

Furthermore, the present invention includes reconstituted plasma that is reconstituted using the reconstitution solution described herein. In particular, the reconstituted plasma includes the reconstitution solution and spray dried plasma, and is obtained by mixing spray dried plasma and the reconstitution solution having a NACNC in the range between about 5 mM and about 20 mM and water. The reconstituted plasma of the present invention has platelet clotting properties (e.g., aggregation and adhesion properties) that are about the same or better, as compared the starting plasma (e.g., FFP).

The present invention also involves a novel assay for determining platelet adhesion and aggregation using microfluidic flow cell system having a shear flow through one or more channels. The method includes the steps of coating a channel with an agent that allows for platelet adhesion (e.g., collagen, gelatin, fibronectin, and the like) to thereby obtain a coated channel, and contacting a sample with the coated channel upon induction of a flow. The sample is either whole blood directly labeled with a dye or detector; or whole blood and an anti-platelet antibody that is indirectly labeled with a detector. The methods then involve inducing a shear flow of the sample through the coated channel; and detecting the directly or indirectly labeled platelets. In an embodiment, the shear flow induced can be an arterial shear rate (e.g., 400 s⁻¹ to 1700 s⁻¹) or a pathological shear rate (e.g., 2000 s⁻¹ to about 20,000 s⁻¹). Platelet function (e.g., fluorescence area (e.g., coverage area of the platelets), fluorescence intensity of the platelets, morphology, or combination thereof) is determined based on data from the detection of the directly or indirectly labeled platelets. Detection of the platelets can be measured periodically (e.g., every 10, 20, 30, 40, 50, 60 seconds) over a time period (e.g., 5, 10, 15, 20, 25, 30, 35, 40, 45, 50, 55, 60 minutes). The whole blood sample can be reconstituted whole blood sample used in the assay to assess the reconstituted plasma. The whole blood sample includes, in an embodiment, reconstituted plasma (e.g., spray dried plasma rehydrated with the reconstitution solution), platelets, and red blood cells. The method includes a step of washing/blocking the coated channels with a buffer. In an embodiment, the reconstituted plasma utilizes the reconstitution solution, described herein, that includes a non-anticoagulant compound that does not bind calcium in the range between about 5 mM and about 20 mM; and water. The platelets and red blood cells for the whole blood can be obtained from a donor and native plasma is removed. In an embodiment, the platelet and red blood cells are combined with reconstituted spray dried plasma and the whole blood has about a 40% hematocrit (e.g., between about 35% hematocrit and 45% hematocrit) and consistent platelet count of 200,000 mm⁻³ (e.g., between about 180,000 mm⁻³ and about 220,000 mm⁻³).

The present invention has numerous advantages. The present invention improves the spray dry plasma process by providing a reconstitution solution that allows the reconstituted plasma to function as good as, if not better than FFP, and provides a novel way of assaying samples for spray dried plasma efficiency. This is a significant advantage since spray dry plasma is easier to store and use in the field. Accordingly, the present invention provides a product that is easier to store and use, but functions as well as or better than the current standard.

### BRIEF DESCRIPTION OF THE DRAWINGS

FIGS. 1A-1F show a series of line and bar graphs proving BIOFLUX system analyses under arterial (normal) shear of rehydrated SpDPs (Spray Dried Plasma) and FFP (Frozen Fresh Plasma) (FIGS. 1A and 1B), or rehydrated SpDPs and FFP mixed with equal volume of platelet poor plasma (shown in FIGS. 1C & 1D), denoted by PPP (Platelet Poor Plasma) with a thrombin-specific inhibitor, D-Phe-L-Pro-L-ArgCH₂Cl (PPACK), and under pathological (trauma) shear of rehydrated SpDPs and FFP (FIGS. 1E and 1F). SpDP/PreT (Spray Dried Plasma that was Pre-Treated): SpDP derived from plasma pretreated with 7.4 mM citric acid, rehydrated in 2.7 mM sodium carbonate; SpDP1: untreated SpDP rehydrated in 7.4 mM citric acid and pH adjusted to corresponding FFP using 0.5 M sodium carbonate stock; SpDP2: untreated SpDP rehydrated in 14 mM glycine HCl; FFP: thawed FFP control. All rehydrated SpDP samples were matched to FFP in protein concentration and pH. FIU (fluorescent intensity unit); Top panel: Time-lapse fluorescence development; Bottom panel: slope of FIU at Final Time Point.

FIG. 2 is a bar graph showing analyses of rehydrated SpDPs and FFP using the Chrono-Log Ristocetin Cofactor Assay. SpDP/PreT: SpDP derived from plasma pretreated with 7.4 mM citric acid, rehydrated in 2.7 mM sodium carbonate; SpDP1: untreated SpDP rehydrated in 7.4 mM citric acid and pH adjusted to corresponding FFP with 0.5 M sodium carbonate; SpDP2: untreated SpDP rehydrated in 14 mM glycine HCl; FFP: thawed FFP control. All rehydrated SpDP samples were matched to FFP in protein concentration and pH.

FIGS. 3A-3B show a series of line graphs showing a BIOFLUX system study of von Willebrand disease (VWD) plasmas in comparison with normal platelet poor plasma (PPP). FIG. 3A shows results under arterial shear and FIG. 3B shows results under pathological shear.

FIGS. 4A-4B depict bar graphs showing aPTT and TEG analysis of SpDP samples in comparison with FFP. SpDP samples were rehydrated in various acidic rehydration solutions matching the pH (˜7.4) and protein concentration of FFP. FIG. 4A is a bar graph showing the R-time (minutes) of SpDP samples reconstituted using ascorbic acid (11.5 mM), citric acid (4.7 mM), gluconic acid (11.6) mM), glycine HCl (11.6 mM) lactic acid (12.6 mM), monosodium citrate (6.5 mM), NaH2PO4 (14.9 mM) and FFP. FIG. 4B is a bar graph showing the aPTT (seconds) of SpDP samples reconstituted using ascorbic acid (11.5 mM), citric acid (4.7 mM), gluconic acid (11.6) mM), glycine HCl (11.6 mM) lactic acid (12.6 mM), monosodium citrate (6.5 mM), NaH2PO4 (14.9 mM) and FFP.

### DETAILED DESCRIPTION OF THE INVENTION

A description of preferred embodiments of the invention follows.

The present invention relates to a novel reconstitution solution for reconstituting spray dried plasma (SpDP). The reconstitution solution of the present invention results in reconstituted spray dried plasma that functions as good as or in some cases better than the starting plasma (e.g., Fresh Frozen Plasma (FFP)). The present invention includes the reconstitution solution, reconstituted spray dried plasma, reconstitution methods and methods for assessing platelet adhesion/aggregation.

The reconstitution solution of the present invention includes a Non-AntiCoagulant that does Not bind Calcium (NACNC). A NACNC as used herein includes any substance such as an acid or acidic salt or other substance that is physiologically compatible for addition to reconstitution solution for spray dried plasma and to the subjects (human or otherwise) to which the reconstituted plasma is to be transfused. The reconstitution solution can have one or more NACNC, and when mixed with spray dried plasma achieves a pH that is comparable with native plasma. In a particular embodiment, NACNC compounds used for reconstitution of SpDP are acidic substances that do not cause pseudo-prolongation of either aPTT (activated Partial Thromboplastin time) or R-time of Thromboelastogram (TEG). aPTT measures the activity of the intrinsic and common pathways of coagulation. The aPTT test is performed on an Instrumentation Laboratory ACL Top coagulation analyzer. NACNC compounds for use in the reconstitution solution of the present invention do not prolong the aPTT of FFP when spiked at 5-20 mM. FFP has an aPTT value between about 20 and about 40 seconds when measured using Instrumentation Laboratory's aPTT-SP assay. Similarly, the R-time of TEG refers to the rate at which an initial clot formation is detected. It is a reflection of the coagulation factor cascade (thrombin generation and fibrin formation) and is tested by Thrombelastograph Hemostasis Analyzer. NACNC compounds for use in the reconstitution solution of the present invention do not prolong the R-time of FFP when spiked at about 5 to about 20 mM. FFP has a R-time between about 5 and about 15 minutes using Kaolin as an activator. To determine if a candidate as a NACNC compound, the R-time and aPTT can be compared to a positive control, such as glycine HCl. A candidate that performs similarly to glycine HCl and is a non-anticoagulant that does not bind calcium can be further tested for use in the reconstitution solution of the present invention. In an embodiment, the R-time and aPTT times of the candidate compounds is within 1% to about 35% (e.g., 1, 5, 10, 15, 20, 25, 30, and 30%) of the R-time and aPTT times of glycine HCl. A candidate compound that satisfies the R-time and aPTT time threshold can be tested as a reconstitution solution using the cell flow assay that induces shear flow, as described herein. These compounds typically include, for example, non-calcium binding acidic substances such as HCl, acetic acid, glycine HCl and ascorbic acid or weak calcium binding acidic substances such as lactic acid and gluconic acid. NACNC can be used in the range of between about 5 mM and about 20 mM (e.g., 5, 6, 7, 8, 9, 10, 11, 12, 13, 14, 15, 16, 17, 18, 19, or 20 mM). In an embodiment, the concentration of NACNC ranges between about 8 and about 16 mM in water. When two or more NACNC compounds are used, in an embodiment, the total concentration of the combined NACNC compounds ranges between about 5 mM and about 20 mM. The rehydration solutions can be made from the off-the-shelf NACNC compound and water to the desired concentration, e.g., between about 5 and about 20 mM. Examples of such NACNC for use in the reconstitution solution include, glycine HCl, ascorbic acid, lactic acid, and gluconic acid. The Examples provided herein show data using glycine HCl as an effective compound in the reconstitution solution of the present invention. Other NACNC compounds that are known in the art, or meet the criteria described herein can be used in the reconstitution solution of the present invention. Additional NACNC compounds can be determined by experimentation to have the criteria described herein.

Additional NACNC compounds, now known or discovered in the future can be used as long as the NACNC is biocompatible, a non-anticoagulant, and does not bind calcium. Coagulation assays are known in the art and can be used to determine if a potential compound is a NACNC compound suitable for use with the reconstitution solution of the present invention. Examples of such assays include, but not limited to aPTT and TEG. A compound that is biocompatible and is negative or exhibits reduced activity as an anticoagulant and calcium binding is candidate for use in the reconstitution solution of the present invention. In an embodiment, the compound can be evaluated using both assays aPTT and TEG.

The reconstitution solution of the present invention includes one or more NACNC compounds in an amount ranging from 5 mM to 20 mM (e.g., about 10 mM to about 14 mM). Other components of the reconstitution solution include water, or other agents if desirable. The reconstitution solution is made from off-the-shelf NACNC chemicals and water to the desired concentration, e.g., between about 5 and about 20 mM. The reconstitution solution can be made in a wide range of volumes, such as 0.1, 1, 5, 10, 100, 1,000, or 10,000 L. The water used in the reconstitution solution can be sterile water (e.g., water for injection (WFI) or similar) or clean, non-sterile water and, if desired, filtered after reconstitution. In an embodiment, the reconstitution solution of the present invention has a pH of about 6.5 and about 8.0, and reconstituted spray dried plasma of the present invention, in an embodiment, has a pH of about 6.8 to about 7.6, or about 6.9 to about 7.5.

The reconstitution solution is used to reconstitute plasma that has been spray dried. The spray drying process, under certain conditions and parameters, can harm the plasma proteins. The spray drying process, depending on the parameters, can reduce amounts of certain large multimeric proteins (e.g., von Willebrand factor (vWF)), reduce large proteins into smaller proteins, and/or affect the activity/functionality of such proteins. The reconstituted plasma of the present invention not only provides proteins that function as well as those FFP but in certain aspects surprisingly improves their functionality.

The reconstitution solution of the present invention is surprising because, if the plasma proteins are somehow damaged, reduced, modified in some way by the spray drying process, then once the damage is done, one would not expect that a reconstitution solution would be able to repair the damaged proteins. In particular, prior to the invention, it was understood that the vWF is a large multimeric protein and needs to be intact in order to be effective in platelet adhesion and aggregation. The spray drying process essentially cuts up the large vWF protein into small proteins (low or intermediate molecular weight proteins or smaller multimers), which were believed to be ineffective or less effective than the fully intact, large multimeric protein version of the vWF. The data described herein show that is not the case. In fact, the data in examples 1 and 2 show that spray dried plasma reconstituted with a solution having NACNC works as well as fresh frozen plasma (FFP) and in some cases better than FFP.

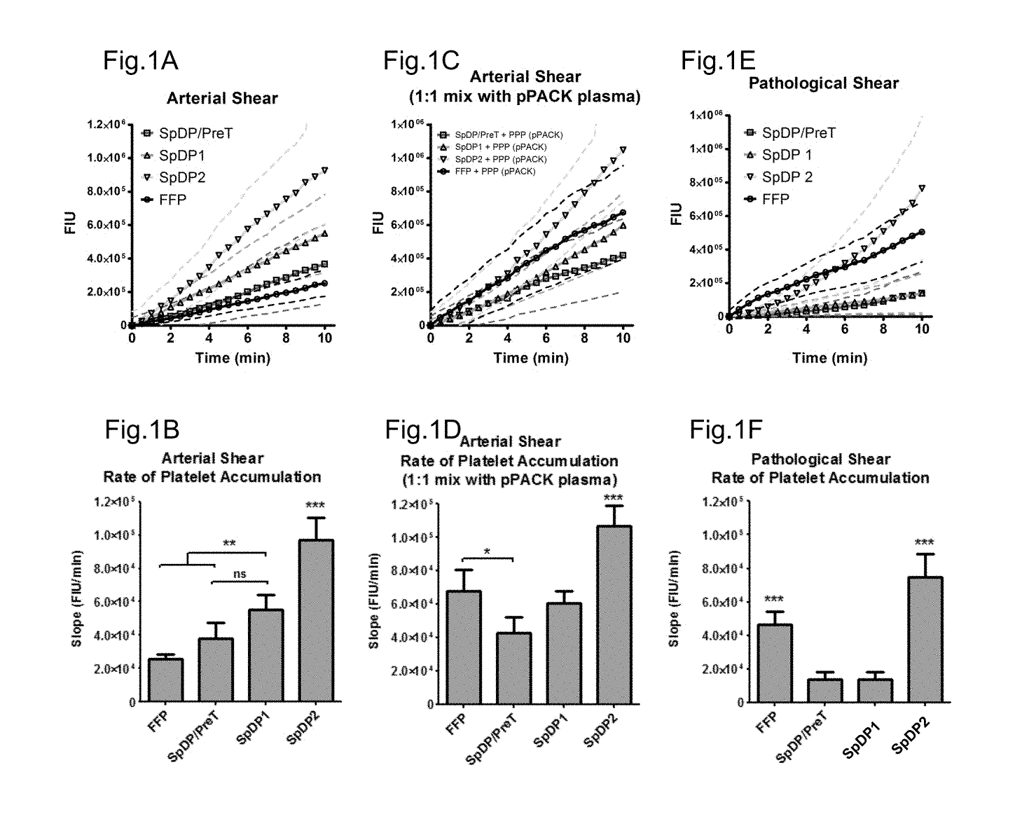

In particular, SpDP samples described in Example 1 outperformed FFP in mediating adhesion and aggregation under normal shear force (FIGS. 1A & B), suggesting the effectiveness of small vWF multimers for platelet adhesion and aggregation. Platelet adhesion and aggregation “mediated” by the reconstituted plasma refers to the measurement of platelet adhesion and aggregation of a sample having reconstituted plasma, platelets and preferably red blood cells. The newly formed small vWF multimers effectively compensated for the loss of large vWF multimers and resulted in equal or better performance of reconstituted spray dried plasma using the reconstitution solution of the present invention. The gain-of-quantity in smaller vWF multimers compensates for the loss-of-quality for mediating platelet adhesion and aggregation found in spray dried plasma. Use of the reconstitution solution makes spray dried plasma comparable to or better than FFP in mediating platelet adhesion and aggregation, key components in clot formation.

von Willebrand factor (vWF) is a large, highly adhesive, multimeric glycoprotein that is present predominantly in plasma (˜85%, produced in endothelial cells) and platelets (˜15%, produced in megakaryocytes). It is important for hemostasis and thrombus formation by acting as a bridging molecule for normal platelet adhesion and aggregation at sites of vascular injury. In addition, vWF functions as a carrier protein for factor VIII (FVIII), thereby protecting FVIII from rapid clearance. Hence, vWF is essential to both primary (platelet-mediated) and secondary (coagulation factor-mediated) hemostasis.

In plasma, vWF exists in a multimeric dimer configuration, ranging in size from, low molecular weight (LMW) dimers to intermediate molecular weight (IMW) and very large, high molecular weight (HMW) dimers. The larger the vWF molecule, the greater the overall number of individual adhesion sites, and thus the greater the overall adhesive capacity. Defects in, or reduced levels of vWF molecules are associated with the von Willebrand disease (VWD).

von Willebrand factor ristocetin cofactor (VWF:RCo) assay is the standard and widely used laboratory test for von Willebrand disease (VWD) diagnosis. It is a functional assay of plasma VWF based upon the degree of platelet agglutination induced after the addition of ristocetin. It measures the interaction of vWF with platelets. Since large vWF multimers are most effective for interactions for platelets, this test is sensitive to the size of vWF multimers and results of this test are described in the Exemplification. It can be implemented in different formats. Automated method improves the assay performance and allows its routine application in comparison with the standard aggregometric method (Chrono-Log Ristocetin Cofactor Assay). VWD plasmas (type 1, 2 &3) have much lower vWF:RCo activity using CHRONO-LOG Ristocetin Cofactor Assay. vWF antigen testing measures the amount of vWF protein, and factor VIII coagulant activity indirectly reflects vWF interaction with factor VIII. vWF multimer analysis visualizes the distribution of vWF multimers and is useful as a reflexive test for subtyping von Willebrand disease (VWD).

SpDP has reduced level of vWF:RCo activity in automated assay format on BCS XP coagulation analyzer, but normal levels of vWF antigen and factor VIII activity, suggesting that spray drying process downsizes vWF multimers, but has no impact on the vWF protein level and the function for binding and stabilizing factor VIII. It also suggests a net increase of vWF molecules in SpDP, i.e., a gain-of-quantity of vWF multimers. vWF multimer analysis confirmed the breakdown of large vWF multimers in SpDP into small ones. Pretreatment of the plasma with citric acid (7.4 mM) prior to spray drying bumps up vWF:RCo activity in SpDP, which has been related to elevated rescue of IMW vWF multimers.

SpDP vWF multimers exhibit similar laddering pattern to Type IIB VWD plasma on multimeric analysis. However, there is a quantitative difference between the two types of plasma. In contrast to VWD plasma which has often low levels of vWF protein (antigen) compared with normal plasma, SpDP plasma has normal level of vWF protein. Importantly, SpDP plasma has higher levels of total vWF multimers than normal plasma. It was determined that with the use of the reconstitution solution of the present invention, the elevated level of small vWF molecules are able to compensate for the reduction of HMW vWF multimers for effective mediation of platelet adhesion and aggregation.

In an embodiment, the reconstitution solution of the present invention demonstrates results of platelet adhesion and aggregation using temperature-controlled flow cell assays such as the BIOFLUX assay performed on the BIOFLUX 1000 system (Fluxion Biosciences, Inc.) (described in the examples), which are comparable to that of FFP. In one aspect, platelet aggregation refers to platelets sticking to one another or clumping together, which is part of the sequence of events leading to the formation of a thrombus or a clot. Platelet adhesion, in another aspect, refers to the ability of platelets to stick to non-platelet surfaces (e.g., collagen surfaces). Specifically, platelet adhesion, in an embodiment, refers to changes in the cell membrane and exposure of molecules that allow for adhesion. Platelet aggregation and adhesion both occur to form a clot. Platelet accumulation, which is a function of both platelet aggregation and adhesion, refers to clot formation and in an embodiment, is measured as a slope generated by collecting fluorescence intensity or area periodically over a time period using the flow cell assay described herein.

Platelet adhesion and aggregation using the flow cell assay is measured, in part, under arterial shear over time, the rate of platelet accumulation. In an embodiment, the reconstituted plasma of the present invention mediates a rate of platelet accumulation that is the same or about the same as that exhibited by the starting plasma, in this case FFP, under an arterial shear. In a particular embodiment, the rate of platelet accumulation (e.g., platelet adhesion and aggregation) using the reconstituted plasma of the present invention is greater by about 1% to about 100% (e.g., by about 10%, 20%, 30%, 40%, 50%, 60%, 70%, 80%, 90%, or 100%), than the rate of platelet accumulation of that in the starting plasma. In another embodiment, the rate of platelet accumulation mediated by the reconstituted plasma of the present invention is at least about 1% to about 4× (e.g., by about 10%, 20%, 30%, 40%, 50%, 60%, 70%, 80%, 90%, 100%, 150%, 200%, 250%, 300%, 350% or 400%) the rate of platelet accumulation of that in the starting plasma (e.g., FFP). Starting plasma is plasma that is not frozen (e.g., never-frozen plasma) or thawed from FFP. In Example 1, the reconstituted plasma of the present invention, prepared in samples with red blood cells and platelets forming whole blood, exhibited rate of platelet accumulation in a range of about more than 4 times the rate of FFP under conditions of arterial shear and about 1.5-2 times the rate of FFP under conditions of pathological shear. (See FIG. 1).

The rate of platelet accumulation indicates how well a clot forms under the shear conditions. The assay of the present invention that induces a shear flow in a channel mimics a human vessel and is able to assess and measure the size and nature of the clot formation over time. This can be measured, in part, by assessing fluorescence of the labeled cells to determine the area of the clot and the intensity of a clot (e.g., three dimensional volume/density) over time. Measurements with BioFlux can be done using the fluorescence intensity of the labeled cells (e.g., platelets). The flow can be adjusted to model normal shear (e.g., arterial shear) or those designed for high shear effects (e.g., pathological shear to mimic conditions such as artery stenosis or a tourniquet) and assess clot formation. In certain experiments, fluorescent images can be collected over time at periodic intervals e.g., such as over a 10 minute period (every 30 seconds for a total of 21 images per run). Platelet accumulation curves can be calculated for a sample using the following metrics: 1. Final coverage area (%) (e.g., indicates the aggregation size); 2. Final fluorescence intensity (in arbitrary units) (e.g., indicates the three dimensional size and density of the clot); and 3. Slope of the lines generated by collecting intensity or area over time at periodic intervals (e.g., every 30 seconds for 10 minutes). The coverage area is the percent florescence measuring the area of space that the clot takes up at a specific location in the vessel. The fluorescence intensity provides an assessment of the density and three-dimensional size and nature of the clot. The slope allows one to assess how quickly the clot forms, and how big and dense the clot gets over time. The absolute numbers shown in the figures will vary based on factors such as donor variability, age of the lamps, alignment of the scope, efficiency of the collagen coating, and the like). Accordingly, control samples are used alongside test sample so that relative comparisons can be performed to assess clot formation and the efficacy of the plasma in a sample (e.g., whole blood samples, and samples that have reconstituted spray dried plasma of the present invention combined with blood cells including red blood cells and platelets).

Platelet adhesion and aggregation and analysis of platelet function can be performed to assess the reconstituted spray dry plasma of the present invention. Performing this analysis under flow is important to understanding the complex biological relationships contributing to hemostasis and thrombosis. The function of platelet receptors and the eventual biological outcome are strongly influenced by fluid shear stress generated by the partially laminar flow of blood in the circulation. Common in vitro methods used in research laboratories to study platelet biology under conditions of shear flow include light transmission aggregometers, cone and plate viscometers, perfusion chambers and more recently, microfluidic flow (perfusion chambers) cells. Perfusion devices, such as parallel plate flow chambers (PPFC) and microfluidic devices, allow similar real-time insight into the dynamic process of platelet adhesion and aggregation behavior. With traditional PPFC, a large blood volume is required and the experimental throughput is especially low (1-2 conditions per hour). This precludes certain experiment types such as murine studies and studies from a single donor that must be performed very quickly after blood collection. The low throughput also prevents the use of a standard parallel-plate flow chamber for population studies. A microfluidic device and control instrument such as BIOFLUX system, which is better suited for platelet adhesion and aggregation assays for single donor studies/testing.

The assay for determining platelet adhesion and aggregation using microfluidic flow cell system having a shear flow is performed with the following steps. Channels of the microfluidic flow cell system are coated with a ligand or cells. The ligand or cells are those to which platelets will adhere. Examples of such ligands include, collagen (e.g., purified, collagen I), fibronectin, gelatin. Examples of cell types include endothelial cells, sub-endothelial cells, and the like. Other ligands or cells suitable for platelet adhesion can be used. The pneumatics of the flow cell system apply a force to push the coating through the channel. In an embodiment, the coating can incubate for a period of time (e.g., 15, minutes, 30 minutes, 45 minutes, 1 hour) before being washed. Channels can then be blocked with a buffer such as bovine serum albumin (BSA). The sample is one that contains platelets and can be obtained and prepared by a method suitable for the particular sample (e.g., whole blood, platelet rich plasma, or platelets). In an embodiment, the sample includes the reconstituted plasma of the present invention combined with red blood cells (e.g., having specific hematocrit) and platelets to form a whole blood sample. In an embodiment, the sample can be platelet rich plasma. The test samples and control samples are labeled directly or indirectly. The whole blood samples can be labeled non-specifically with a detector or dye such as Calcein AM, Celltracker Green (CMFDA), alamarBlue, PKH Cell Linker, and others known in the art. Samples can also be labeled indirectly through the use of one or more fluorescently conjugated anti-platelet antibodies. The inventive platelet assay can use antibodies reactive with platelets, portions of platelets or platelet markers. In a preferred embodiment, the antibodies specifically bind with platelets or portion thereof. An anti-platelet antibody includes monoclonal and/or polyclonal antibodies, or mixtures thereof. In an embodiment, the sample is contacted with an antibody having specificity for the platelet under conditions suitable for formation of a complex between an antibody and platelets. The method can involve contacting or staining the samples with a composition comprising an anti-platelet antibody, having a fluorescent label, under conditions suitable for the formation of labeled complexes between said antibody and activated platelets. Once the sample is ready, the sample is loaded (e.g., into an inlet well of the plate) and the flow of the sample through the channel is induced establishing a shear flow. Pneumatic pressure (precalculated based on viscosity of the sample) is applied to generate a specific shear effect within the geometry of the channel. Settings of the flow to create arterial shear or pathological shear are done on the microfluidic flow cell system such as the BIOFLUX system. In an embodiment, the peak systolic arterial shear rate ranges from about 400 s⁻¹ to 1700 s⁻¹, while pathological shear in stenotic vessels can be from about 2,000 s⁻¹ to about 20,000 s⁻¹ (e.g., 3,000, 4,000, 5,000, 6,000, 7,000, 8,000, 9,000, 10,000, 11,000, 12,000, 13,000, 14,000, 15,000, 16,000, 17,000, 18,000, 19,000 s⁻¹). The conditions of the vessel can change the pathological shear rate and the pathological shear rate can depend on where the observation was made (e.g., right in the neck of the (partially) occluded area will have a tremendous shear effect, but in the recirculation zone the shear rate might be lower). Depending on the particular vessel, in an embodiment, the pathological shear rate can be about 0 to about 100,000 s⁻¹. The settings for arterial shear and/or pathological shear (e.g., to mimic artery stenosis or tourniquet conditions) can be set on the system per manufacturer instructions (e.g., using the BIOFLUX interface and controller software, see Bioflux 1000Z User Giude, Doc #630-0070 Rev A Fluxion Biosciences Inc. South San Francisco Calif. (Jan. 10, 2011), the teachings of which are incorporated herein by reference). Once the flow is induced, the detection of fluorescence can be performed by detecting or measuring (directly or indirectly) the formation of a complex or clot. In an embodiment, a fluorescence detection camera on a microscope captures images within the channel, allowing for visualization and quantification of the fluorescently labeled platelets as they adhere to the collagen surface and begin to aggregate. Fluorescence detection can occur periodically (e.g., every 10, 20, 30, 40, 50, 60 seconds) over a time period (e.g., 5, 10, 15, 20, 25, 30, 35, 40, 45, 50, 55, 60 minutes) to capture time lapse microscopy data. Adhesion and aggregation size, intensity, morphology, and the like are analyzed across multiple fields of view per condition.

In order to test the functionality of reconstituted spray dried plasma of the present invention, whole blood was collected from donors and centrifuged to enable collection of individual components. Both platelets and red cells were washed to remove as much native plasma as possible, and a simulated whole blood product using freshly washed platelets, red cells, and reconstituted spray dried plasma or control plasma was constructed with a 40% hematocrit (e.g., between about 35% hematocrit and 45% hematocrit) and consistent platelet count of 200,000 mm⁻³ (e.g., between about 180,000 mm⁻³ and about 220,000 mm⁻³).

For example, a description of the BIOFLUX system and methodology can be found in US Patent publication No. 20120264134, 20070243523 and U.S. Pat. No. 8,293,524, and published PCT Application No. WO/2007/117987, the entire teachings of which are incorporated herein by reference.

While many studies have been conducted using the BIOFLUX system which observe the effects of altered platelet function, this current application is novel in that it revolves around observing the contributions of the plasma, and in particular the reconstituted spray dried plasma product (SpDP) of the present invention. Because the adhesion of platelet to collagen is facilitated through molecular mechanisms involving the plasma protein von Willebrand Factor (vWF), and because SpDP has been demonstrated to have a deficiency of high molecular weight multimers of vWF (caused by the spray drying process), the ability of SpDP (in comparison to standard fresh frozen plasma, FFP) to negotiate platelet-to-collagen binding have been examined with the microfluidic flow cell system in this novel fashion.

### Storage and Reconstitution

Once the plasma is dried, it can be stored for a period of time until a patient is in need thereof. In an embodiment, the spray dried formulated plasma can be stored at room temperature, refrigerated temperature, or even in certain cases at higher temperatures. In one aspect, the spray dried plasma is kept between about room temperature (between about 20° C. and 25° C.) and 37° C. As used herein, chilling refers to lowering the temperature of the spray dried plasma to a temperature that is less than about 25° C. In some embodiments, the spray dried plasma is chilled to a temperature that is less than about 15° C. In some preferred embodiments, the spray dried plasma is chilled to a temperature ranging from between about 0° C. to about 4° C. Chilling also encompasses freezing the spray dried plasma, i.e., to temperatures less than 0° C., 20° C., 50° C., and 80° C. or cooler. In some embodiments, the spray dried plasma is stored at room temperature for a period between 1 day and 30 days (e.g., 1, 2, 3, or 4 weeks). For example, the spray dried plasma is stored for at least 4, 5, 6, 7, 8, 9, 10, 11, 12, 13, 14, 15, 16, 17, 18, 19, 20, 21, 22, 23, 24, 25, 26, 27, and 28 days or longer.

The methods of the present invention include reconstituting (mixing or combining) the reconstitution solution with spray dried plasma to form the reconstituted plasma. Once the plasma is dried, the plasma is reconstituted or rehydrated so that it can be transfused into a patient. One unit of SpDP can be reconstituted with the reconstitution solution of the present invention at a volume ranging between about 30% and 100% (e.g., 30, 40, 50, 60, 70, 80, 90, and 100%) of the volume of the starting plasma. For example, SpDP manufactured from 240 mL of FFP can be rehydrated in 80, 150, 200, 240 mL of reconstitution solution. A sterile connection between the unit of dried plasma and the reconstitution solution is made and the reconstitution solution is inserted/injected into the unit of dried plasma, and mixed or shaken to obtain a uniform reconstituted unit of plasma.

In an embodiment, the methods include selecting a subject in need of plasma and transfusing a reconstituted plasma unit of the present invention to the subject in need of plasma. Patients can be transfused with one or more units of reconstituted plasma, depending on the patient's need. Once reconstituted, the plasma should be transfused contemporaneously into a patient or within a period of time ranging from about 0 to about 4 hrs (e.g., 15 minutes, 30 minutes, 45 minutes, 1, 2, 3, 4 hours) of being reconstituted. Generally, such transfusion/administration can be performed intravenously.

### Plasma

Generally, plasma is the fluid that remains after blood has been centrifuged (for example) to remove cellular materials such as red blood cells, white blood cells and platelets. Plasma is generally yellow-colored and clear to opaque. Blood that is donated and processed to separate the plasma from the other certain blood components, and not frozen is referred to as “never-frozen” plasma. Plasma that is frozen within 8 hours to temperatures, described herein, is referred to herein as “fresh frozen” plasma. It contains the dissolved constituents of the blood such as proteins (6-8%; e.g., serum albumins, globulins, fibrinogen, etc.), glucose, clotting factors (clotting proteins), electrolytes (Na⁺, Ca²⁺, Mg²⁺, HCO₃⁻, Cl⁻, etc.), hormones, etc. Whole blood (WB) plasma is plasma isolated from whole blood with no added agents except anticoagulant(s). Citrate phosphate dextrose (CPD) plasma, as the name indicates, contains citrate, sodium phosphate and a sugar, usually dextrose, which are added as anticoagulants.

The plasma reconstituted with the solution of the present invention can be dried after pooling or unit-by-unit. Pooling of multiple plasma units has some benefits. For example, any shortfall in factor recovery on an equal-volume basis can be made up by adding volume from the pool to the finished product. There are negative features as well. Making up volume from the pool to improve factor recovery is expensive. Importantly, pooled plasma must be constantly tested for pathogens as any pathogens entering the pool from, for example, a single donor, runs the risk of harming hundreds or thousands of patients if not detected. Even if detected, pathogen contamination of pooled plasma would render the whole pool valueless. Testing can be obviated by pathogen inactivation of the plasma by irradiation or chemically such as solvent detergent treatment; however, each such treatment adds cost and complexity to pooled plasma processing. In any event, pooled plasma processing is generally unsuitable to the blood centers and generally only really suitable to an industrial, mass production environment.

Conversely, unit-by-unit (unit) collection and processing is well-suited to the blood center environment and reduces the risk of pooled plasma pathogen contamination by allowing for pre-processing testing for pathogens and tracking of the unit to ensure that each unit leaves the blood center site pathogen free. The inventors have discovered that reconstitution of dried plasma using the reconstitution solution of the present invention results in effective platelet adhesion and aggregation as measured using a flow cell assay. Such efficiency is also very helpful in the pooled plasma environment as well.

### Spray Dryer and the Spray Drying Process

In general, a spray dryer system (spray dryer device) is provided for spray drying a liquid sample such as blood plasma. In an embodiment, the spray dryer system used to spray dry plasma for reconstitution by the solution of the present disclosure includes a spray dryer device and a spray dryer assembly. The spray dryer device is adapted, in an aspect, to receive flows of an aerosolizing gas, a drying gas, and plasma liquid from respective sources and coupled with the spray dryer assembly. The spray dryer device can further transmit the received aerosolizing gas, drying gas, and plasma to the spray dryer assembly. Spray drying of the plasma is performed in the spray dryer assembly under the control of the spray dryer device. Any suitable spray drying system can be used to dry plasma for use in with present invention. For exemplification, a suitable spray dryer is described below.

In certain embodiments, the spray dryer assembly includes a sterile, hermetically sealed enclosure body and a frame to which the enclosure body is attached. The frame defines first, second, and third portions of the assembly, separated by respective transition zones. A drying gas inlet provided within the first portion of the assembly, adjacent to a first end of the enclosure body.

A spray drying head is further attached to the frame within the transition zone between the first and second portions of the assembly. This position also lies within the incipient flow path of the drying gas within the assembly. During spray drying, the spray drying head receives flows of an aerosolizing gas and plasma and aerosolizes the plasma with the aerosolizing gas to form an aerosolized plasma. Drying gas additionally passes through the spray drying head to mix with the aerosolized plasma within the second portion of the assembly for drying. In the second portion of the assembly, which functions as a drying chamber, contact between the aerosolized plasma and the drying gas causes moisture to move from the aerosolized plasma to the drying gas, producing dried plasma and humid drying gas.

In alternative embodiments, the aerosolizing gas can be omitted and the spray dryer assembly head may include an aerosolizer that receives and atomizes the flow of plasma. Examples of the aerosolizer may include, but are not limited to, ultrasonic atomizing transducers, ultrasonic humidified transducers, and piezo-ultrasonic atomizers. Beneficially, such a configuration eliminates the need for an aerosolizing gas, simplifying the design of the spray dryer device and assembly and lowering the cost of the spray dryer system.

The spray drying head in an embodiment is adapted to direct the flow of drying gas within the drying chamber. For example, the spray drying head includes openings separated by fins which receive the flow of drying gas from the drying gas inlet. The orientation of the fins allows the drying gas to be directed in selected flow pathways (e.g., helical). Beneficially, by controlling the flow pathway of the drying gas, the path length over which the drying gas and aerosolized blood plasma are in contact within the drying chamber is increased, reducing the time to dry the plasma.

The dried plasma and humid drying gas subsequently flow into the third portion of assembly, which houses a collection chamber. In the collection chamber, the dried plasma is isolated from the humid drying gas and collected using a filter. For example, the filter in an embodiment is open on one side to receive the flow of humid air and dried plasma and closed on the remaining sides. The humid drying gas passes through the filter and is exhausted from the spray dryer assembly.

In alternative embodiments, the filter is adapted to separate the collection chamber into two parts. The first part of the collection chamber is contiguous with the drying chamber and receives the flow of humid drying gas and dried plasma. The dried plasma is collected in this first part of the collection chamber, while the humid air passes through the filter and is exhausted from the spray dryer assembly via an exhaust in fluid communication with the second part of the spray dryer assembly.

After collecting the dried plasma, the collection chamber is separated from the spray dryer assembly and hermetically sealed. In this manner, the sealed collection chamber is used to store the dried plasma until use. The collection chamber includes a plurality of ports allowing addition of the reconstitution solution of the present invention to the collection chamber for reconstitution of the blood plasma and removal of the reconstituted blood plasma for use. The collection chamber can further be attached to a sealed vessel containing the reconstitution solution for reconstitution.

When handling transfusion products such as blood plasma, the transfusion products must not be exposed to any contaminants during collection, storage, and transfusion. Accordingly, the spray dryer assembly, in an embodiment, is adapted for reversible coupling with the spray dryer device. For example, the spray dryer assembly is coupled to the spray dryer device at about the drying gas inlet. Beneficially, so configured, the spray dryer assembly accommodates repeated or single use. For example, in one embodiment, the spray dryer assembly and spray drying head is formed from autoclavable materials (e.g., antibacterial steels, antibacterial alloys, etc.) that are sterilized prior to each spray drying operation. In an alternative embodiment, the spray dryer head and spray drying chamber is formed from disposable materials (e.g., polymers) that are autoclaved prior to each spray drying operation and disposed of after each spray drying operation.

Apparatuses and methods for spray drying are known in art. Spray drying methods and apparatus are further described in U.S. Pat. Nos. 8,469,202, 8,533,971, 8,407,912, 8,595,950, 8,601,712, 8,533,972, 8,434,242, US Patent Publication Nos. 2016/0082044, 20160084572, 2010/0108183, 2011/0142885, 2013/0000774, 2013/0126101, 2014/0083627, 2014/0083628, and 2014/0088768, the entire teachings of which are incorporated herein by reference.

The parameters for spray drying may include a mechanical drying chamber utilizing a plastic/filter collection bag, a 19G Buchi Mechanical nozzle, a plasma fluid flow rate of 10 mL/min, an aerosol gas flow rate of 20 L/min, an initial drying gas temperature of 125° C., a drying gas flow rate of 550-750 L/min, and a drying gas exhaust temperature of 52° C. Conversely, a Velico Medical alpha model spray dryer may be employed at the same or similar parameters.

The present invention relates to a plasma bag or container for use in reconstituting plasma. The plasma bag or container has at least two compartments or containers. One compartment holds or stores the spray dried plasma and the other container stores the reconstitution solution, as described herein. A tube or connector connects the two compartments and has a frangible barrier. In use, the health care professional can break the frangible barrier to allow the reconstitution solution from one container to travel to the container having the dried plasma. The reconstitution solution mixes with the spray dried plasma in the plasma container and is reconstituted.

### EXEMPLIFICATION

### Example 1: Evaluation of SpDP-Mediated Platelet Adhesion and Aggregation by BIOFLUX Assay

The microfluidic flow cell assay of the present invention, also referred herein as the BIOFLUX assay, is an assay that provides physiologically robust modeling of blood (including both hemostasis and proper cellular function) requires the presence of an environment under flow. Platelets act primarily under flow conditions; following wounding, activating factors are released to induce platelet adhesion to the exposed collagen scaffolding. BIOFLUX System (Fluxion Biosciences, South San Francisco, Calif. 94080) allows the platelet adhesion and aggregation assays to be performed under flowing conditions that mimic those in the human body. This assay uses whole blood, reconstituted from RBC, fluorescently-labelled platelets and plasma, which is perfused through collagen-coated microfluidic channels. The platelet adhesion and aggregation can be monitored by fluorescent microscope.

Experimental Design/Methods

Sample Preparation

ABO-identical FFP units were pooled and split: FFP Control (FFP/CP), untreated plasma for spray dried plasma (untreated SpDP), and plasma pretreated with 7.4 mM citric acid prior to spray drying (SpDP/PreT). SpDP/PreT is rehydrated in 2.7 mM sodium carbonate (SpDP/PreT). Untreated SpDP was rehydrated in 7.4 mM citric acid (SpDP1) to match the citrate level in SpDP/PreT and the pH was adjusted to match corresponding FFP/CP with 0.5 M sodium carbonate solution. In another arm, untreated SpDP was rehydrated in 14 mM glycine HCl to match the citrate level in FFP (SpDP2). All SpDP samples were adjusted for protein and pH (Na₂CO₃) to match closely with FFP.

Citrate: a total of 4 test samples were prepared from each FFP pool: FFP/CP, SpDP1, SpDP2 and SpDP/PreT. The citrate concentration in SpDP/PreT was similar to that of SpDP1. The citrate concentration in SpDP2 was comparable to FFP/CP as they both lacked the additional 7.4 mM citric acid that was added to SpDP/PreT and SpDP1. The BIOFLUX assay is sensitive to citrate concentration.

vWF: all samples had similar levels of vWF protein. FFP had normal size distribution of vWF multimers. SpDP1 and SpDP2 were identical, with reduced HMW and IMW vWF multimers, but elevated LMW vWF multimers. SpDP/PreT had reduced HMW vWF multimers, about normal IMW vWF multimers, elevated levels of LMW vWF multimers compared with FFP, and reduced levels of LMW vWF multimers compared with SpDP1/SpDP2. The total molar concentration of vWF multimers in the test samples was SpDP1=SpDP2>SpDP2/PreT>FFP with a reversed order for large vWF multimers, SpDP1=SpDP2<SpDP2/PreT<FFP. The citrate concentration was FFP=SpDP2<SpDP1=SpDP/PreT (contains 7.4 mM more than FFP & SpDP2).

Spray Drying Conditions:
Drying chamber: Mechanical (PN00511)
Collection method: Plastic/filter bag (PN01080)
Collection Bag Constraint PN01172
Nozzle: 19 G Buchi Mechanical
Plasma fluid flow rate: 10 mL/minute
Aerosol gas flow rate: 20 L/minute
Drying gas initial temperature: 125° C.
Drying gas flow rate: 550-750 L/min
Drying gas exhaust temperature: 52° C.

### Detailed Procedure Using BIOFLUX Assay is Outlined as Follows:

1. Introduce 25 μL of coating (100 μg/ml collagen, 100 μg/mL fibronectin, or 1% gelatin as required) into outlet wells of BIOFLUX plate and set the pump to generate a shear stress of 0.5 dyn/cm² to induce flow until the fluid front reaches the end of microscope viewing area. Let the coating incubate on the plate for 1 hr in the biosafety cabinet.
2. Wash by adding 300 μL of 20% BSA into the inlet wells and generating flow with shear stress of 5 dyn/cm² for 3 min. Rinse with 300 μL of PBS in the same manner. PBS is aspirated and an equal amount of PBS is added to all desired wells to remain until use.
3. Platelets (either in platelet-rich plasma or washed and pelleted) are adjusted to an appropriate concentration with platelet-poor plasma and labeled by incubation with 1 μM Calcein AM for 30 min in the dark at 37° C. After incubation, these labeled platelets are mixed with red blood cells (typically from the same donor as the platelet source) to a hematocrit of 40%.
4. A sample volume of 400 μL is transferred to the inlet wells of the BIOFLUX plate; region of interest and objective focus are quickly confirmed for each microchannel's viewing area. Shear rates are set to the desired level and images are acquired every 30 seconds for a period of 10 min.

After completion, images are processed with Montage by setting the background threshold and analyzing percent surface coverage and integrated fluorescence intensity. These values are exported to GraphPad Prism for compilation and statistical analysis

Results

Below are the results for the area of the viewing window which is fluorescent and directly correlated to the number of platelets adhered. The mean value is in bolded.

BioFlux - Arterial Shear - Area Coverage (%)

| | FFP | SpDP/PreT | SpDP 1 | SpDP 2 |
|---|---|---|---|---|
| Minimum | 1.868 | 1.251 | 3.852 | 16.95 |
| 25% Percentile | 8.973 | 4.09 | 5.334 | 30.36 |
| Median | 14.03 | 11.53 | 15.24 | 57.9 |
| 75% Percentile | 17.88 | 29.38 | 28.39 | 76.98 |
| Maximum | 21.16 | 40.01 | 56.55 | 81.06 |
| Mean | 13.34 | 16.03 | 19.9 | 54.19 |
| Std. Deviation | 5.711 | 13.77 | 17.04 | 24.42 |
| Std. Error | 1.806 | 4.353 | 5.39 | 7.722 |
| of Mean | | | | |
| Lower 95% CI | 9.257 | 6.182 | 7.708 | 36.72 |
| of mean | | | | |
| Upper 95% CI | 17.43 | 25.88 | 32.09 | 71.66 |
| of mean | | | | |

BioFlux - Pathological Shear - Area Coverage (%)

| | FFP | SpDP/PreT | SpDP 1 | SpDP 2 |
|---|---|---|---|---|
| Minimum | 0.05556 | 0.009167 | 0.0225 | 0.01639 |
| 25% Percentile | 0.4967 | 0.03188 | 0.03174 | 0.223 |
| Median | 3.811 | 0.215 | 0.311 | 1.197 |
| 75% Percentile | 7.898 | 1.091 | 0.9272 | 7.237 |
| Maximum | 11.92 | 6.643 | 4.794 | 18.44 |
| Mean | 4.206 | 1.067 | 0.8172 | 4.347 |
| Std. Deviation | 4.094 | 2.067 | 1.45 | 6.911 |
| Std. Error | 1.295 | 0.6535 | 0.4585 | 2.186 |
| of Mean | | | | |
| Lower 95% CI | 1.277 | −0.4114 | −0.22 | −0.5971 |
| of mean | | | | |
| Upper 95% CI | 7.134 | 2.545 | 1.854 | 9.291 |
| of mean | | | | |

The above results correlate with the fluorescent intensity shown in FIG. 1. The fluorescent intensity unit (FIU) time lapse reflects how the intensity (corresponding to adherent platelets) increases over time in the various samples. The slope is calculated from the linear regression line and gives a picture of which samples are having a better adhesion response over time.

All SpDP samples outperformed FFP in mediating adhesion and aggregation under normal shear force (FIGS. 1A & B), suggesting the effectiveness of small vWF multimers for platelet adhesion and aggregation. Small vWF multimers are not as effective as large vWF multimers on a one-to-one basis in mediating platelet adhesion. However, the breakdown of large vWF multimers resulted in a loss of quality but brought a gain of quantity to the small vWF multimers; it was surprising and unexpected to find that the team work of newly formed small vWF multimers effectively compensates for the loss of large vWF multimers. The better performance of SpDP2 than SpDP1 highlighted the possible interference of citrate with the assays.

Mixing of test samples 1:1 with citrate-free platelet poor plasma (containing pPACK as anticoagulant) reduced the citrate concentration by 50% in all samples (FIGS. 1C & D), led to a 50% (relative to the starting plasma) compensation for the lost HMW vWF multimers in all SpDP samples, which still had higher than normal levels of IMW and LMW vWF multimers, but had no impact on FFP in terms of vWF. Indeed, the plasma mixing appeared to have corrected the interference of citrate in FFP, but had little impact on SpDP samples for platelet adhesion although the mixing not only brought down the citrate concentration, but also dramatically increased HMW vWF multimers. Together, the data indicates that fragmentation of HMW vWF multimers to LMW vWF multimers during spray drying of the plasma does not impair the function of the plasma in promoting platelet adhesion and aggregation.

Under pathological shear force, the high citrate-containing SpDP/PreT and SpDP1, were much less effective than the low-citrate containing FFP and SpDP2 in mediating platelet adhesion, suggesting the more severe interference of citrate with the assay. The gain-of-quantity of vWF multimers in SpDP/PreT and SpDP1 cannot overcome the interference of the citrate. However, the benefit of gain-of-quantity of vWF multimers remained visible by comparing SpDP2 with FFP. SpDP2 was better than FFP.

In summary, the gain-of-quantity in vWF multimers compensates for the loss-of-quality for mediating platelet adhesion and aggregation by SpDP. SpDP is comparable to FFP in mediating platelet adhesion and aggregation.

Conclusion

SpDP rehydrated in glycine HCl functions at least as effective as FFP in supporting platelet adhesion under both normal and pathological conditions.

### Example 2: Von Willebrand Ristocetin Cofactor Activity in Rehydrated SpDP

The von Willebrand Ristocetin Cofactor (vWF:RCo) Assay is an in vitro assay that can assess the ability of plasma, in the presence of Ristocetin to induce platelet agglutination. The aggluintation is initiated by the ristocetin, which mediates the binding of vWF to the platelet receptor glycoprotein Ib (GpIb). The rate at which platelet agglutination occurs correlates to the concentration and functionality of circulating vWF in the plasma. The platelets utilized for vWF:RCo assay are fixed, as to prevent the secretion of vWF from platelet alpha granules, ensuring only circulating vWF is evaluated.

Experimental Design/Methods

Chrono-log Ristocetin Cofactor Assay was performed following manufacturer's instructions (Stago BNL, The Netherlands) and summarized as follows:
1. Reconstitute SpDP samples; thaw FFP rapidly
2. Prepare standards per manufacturer's instructions using reference plasma (supplied in kit)
3. Dilute test samples 1:1 with tris buffered saline (TBS; supplied in kit); mix well
4. Reconstitute the lyophilized platelets (supplied in kit) with TBS
5. Prepare blank sample by mixing 1:1 TBS with reconstituted platelets
6. Place cuvettes into Chrono-log warming chamber along with a stir bar
7. Add 0.4 mL reconstituted platelets to cuvette
8. Add 0.05 mL Ristocetin (supplied in kit) to cuvette; mix well
9. Move cuvette to aggregometer chamber and incubate for 2 min
10. Set 0% and 100% baselines on sample per manufacturer's instructions
11. Add 0.05 mL of the first standard and record data
12. Repeat steps 6-11 for all remaining standards and samples
13. Collect slope data from Chrono-log and prepare a standard curve on a log-log fit
14. Calculate Ristocetin Cofactor Activity levels of samples based on standard curve

Results

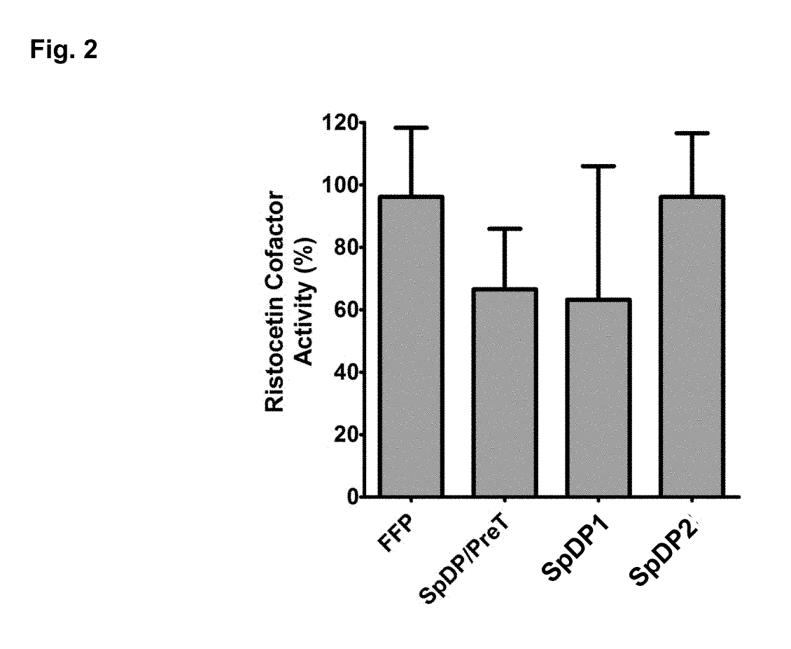

The results are shown in FIG. 2.

### Example 3: BIOFLUX Study of VWD Plasmas Compared with Normal FFP

To confirm the specificity/sensitivity of the microfluidic flow cell assay of the present invention with respect to vWF function, VWD type 1, 2 & 3 plasmas (obtained from Biomed) were evaluated for promoting adhesion of platelets to collagen in comparison with FFP. Type 3 VWD is characterized by severe plasma VWF deficiency, Type 2 has functionally deficient plasma VWF and Type 1 has reduced (below normal) levels of plasma VWF, which is functionally essentially normal.

Experimental Design

Whole blood from healthy donors was collected into citrate tubes. Platelets, RBCs and plasma (platelet poor plasma, PPP) were prepared by centrifugations. Platelets were washed, resuspended in PPP or VWD plasmas (Type 1, 2 and 3), and labeled with calcein-AM. RBCs were also washed. ‘Whole blood’ was then reconstituted from platelet suspension, washed RBCs and corresponding plasmas, and analyzed by BIOFLUX system.

### Results:

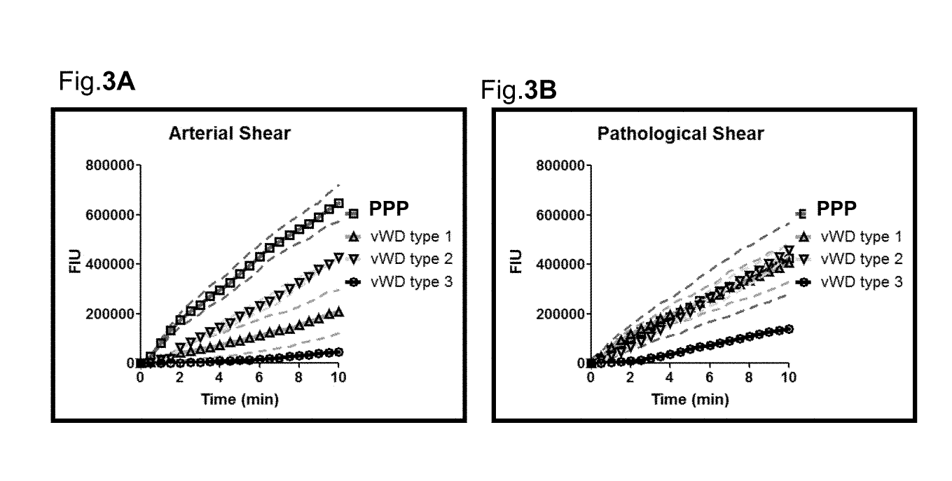

VWD results are shown in FIG. 3. Type 3 plasma was significantly worse than FFP in platelet accumulation; for normal shear, the VWD Type 1 and Type 2 plasmas were also much deficient at facilitating platelet adhesion in comparison to PPP. In the pathological shear, one of the PPP samples was much lower than the other (n=2), which skewed those results resulting in PPP being about the same as VWD Type 1 and Type 2. The data confirmed the specificity/sensitivity of the BIOFLUX assay for evaluating the function of vWF multimers, although less sensitive under pathological shear. This conclusion supports the aforementioned findings regarding SpDP performing as good as or better than PPP when reconstituted with glycine-HCl under both arterial and pathological shear rates in facilitating platelet adhesion to a collagen surface under flow.

### Example 4. Screening for Suitable Acidic Substance for Preparing Rehydration Solution

aPTT and TEG are readily available assays that can be used for the initial screening for the suitable acidic substance for the preparation of SpDP rehydration solution. Confirmation of the candidate compound in the presence of platelets using assays described herein is needed.

Spray drying of plasma leads to CO₂ loss and results in an alkaline plasma product if the spray dried plasma is reconstituted in water. The alkaline plasma product may have deficient function and stability. An acidic rehydration solution that produces plasma product with neutral pH is desirable in this regard. The following outlines the procedure for identifying suitable acidic substance(s) for preparing rehydration solution.

One unit of spray dried plasma was dissolved in water to about 75% of the plasma volume prior to spray drying (˜300 mL), and the protein concentration was measured using NanoDrop 1000. The plasma solution was diluted with water to match the protein concentration of the starting plasma FFP prior to spray drying. The reconstituted plasma solution was split to 20-mL aliquots. Each aliquot was titrated with a stock solution of the acidic substance, and monitored pH readings with pH meter. The pH value and volume of stock solution of the acidic substance added to the plasma were recorded after the reading stabilized. A titration curve was then generated in Excel. The concentration of each acidic substance in the rehydration solution was obtained from the curve, which was used to prepare the rehydration solution that gave rehydrated SpDP ˜pH 7.4 when the protein concentration is matched to corresponding FFP.

Rehydration solutions were then prepared and aliquots of SpDP powder were then rehydrated matching the protein concentration of FFP and had ˜pH 7.4. These samples were analyzed by aPTT and TEG in comparison with FFP. The aPTT values and TEG R-times were compared among the samples.

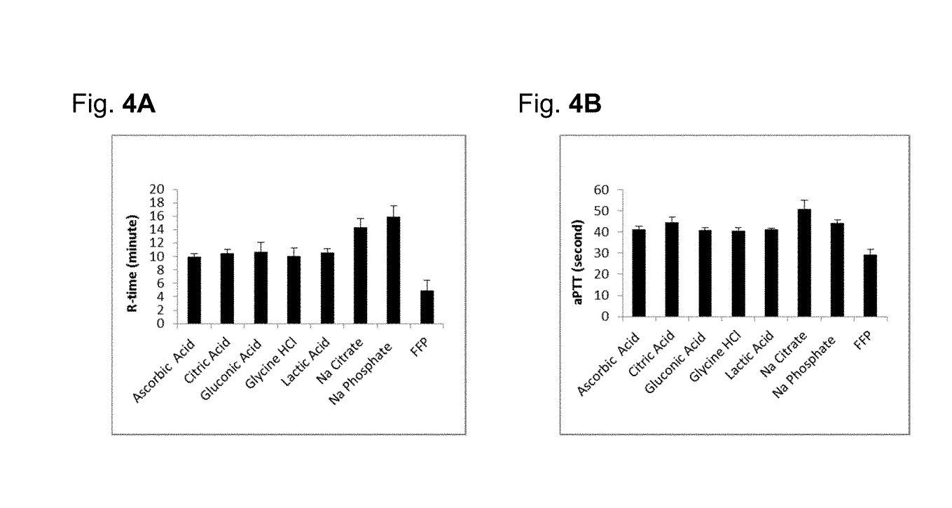

FIGS. 4A-4B show aPTT and TEG analysis of SpDP samples in comparison with FFP. SpDP samples were rehydrated in various acidic rehydration solutions matching the pH (˜7.4) and protein concentration of FFP. FIG. 4A shows a bar graph of SpDP samples reconstituted using ascorbic acid (11.5 mM), gluconic acid (11.6) mM), glycine HCl (11.6 mM) or lactic acid (12.6 mM) had similar TEG R times of about 10 min. Anticoagulant citric acid at 4.7 mM (converted to 4.7 mM citrate at neutral pH) did not prolong TEG R-time. However, about 2 mM increase of the citrate (6.5 mM monosodium citrate) significantly prolonged the R-time compared with glycine HCl (P-value: 0.017). NaH₂PO₄ (14.9 mM) had significantly prolonged R-time compared with glycine HCl (P-value: 0.008). The R-time prolongation in monosodium citrate and NaH2PO4 can be attributed to the complexation/binding of calcium by citrate or phosphate. All pH-adjusted SpDP samples had longer R-time than FFP.

FIG. 4B shows SpDP samples reconstituted using ascorbic acid, gluconic acid, glycine HCl and lactic acid had similar aPTT. Citric acid (4.7 mM) appeared to prolong aPTT although not significantly from glycine HCl (P˜0.069). Similar to TEG, SpDP samples reconstituted from monosodium citrate (6.5 mM) and NaH₂PO₄ (14.9 mM) had significantly prolonged R-time compared with glycine HCl (P-value: 0.014 for monosodium citrate, 0.046 for NaH₂PO₄). All pH-adjusted SpDP samples had longer aPTT than FFP.

TEG and aPTT data showed that ascorbic acid, gluconic acid, glycine HCl and lactic acid do not appear to interfere with coagulation assays, and are worthy of further evaluation in the presence of platelets, e.g. in Bioflux assay. The outstanding property of gluconic acid is its excellent chelating power in alkaline solutions. However, at ˜pH 7.4 it does not appear to have a significant impact of the coagulation assays.

The terms about, approximately, substantially, and their equivalents may be understood to include their ordinary or customary meaning. In addition, if not defined throughout the specification for the specific usage, in an embodiment, these terms can be generally understood to represent values about but not equal to a specified value. For example, 1%, 0.9%, 0.8%, 0.7%, 0.6%, 0.5%, 0.4%, 0.3%, 0.2%, 0.1%, 0.09% of a specified value.

Ranges of values include all values not specifically mentioned. For example, a range of “20% or greater” includes all values from 20% to 100% including 35%, 41.6%, 67.009%, etc., even though those values are not specifically mentioned. The range of 20% to 30% shall include, for example, the values of 21.0% and 28.009%, etc., even though those values are not specifically mentioned.

The terms, comprise, include, and/or plural forms of each are open ended and include the listed items and can include additional items that are not listed. The phrase “and/or” is open ended and includes one or more of the listed items and combinations of the listed items.

The relevant teachings of related applications, U.S. Provisional Application No. 62/319,584, entitled, “Reconstitution Solution For Spray-Dried Plasma” by Qiyong Peter Liu et al., filed Apr. 7, 2016; U.S. Provisional Application No. 62/319,651, entitled, “Reconstitution Solution For Spray-Dried Plasma” by Qiyong Peter Liu et al., filed Apr. 7, 2016; and U.S. application Ser. No. 15/481,573, entitled, “Reconstitution Solution For Spray-Dried Plasma” by Qiyong Peter Liu et al., filed Apr. 7, 2017, are incorporated herein by reference in their entirety. The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1) A reconstitution solution for use in reconstituting spray dried plasma, wherein the spray dried plasma is dried from starting plasma obtained from a donor, the reconstitution solution comprises:
a) at least one acidic non-anticoagulant compound that does not bind calcium in the range between about 5 mM and about 20 mM; and
b) water;
wherein the reconstitution solution is added to spray dried plasma to obtain reconstituted plasma.

2) The reconstitution solution of claim 1, wherein the starting plasma that is a never-frozen plasma or thawed from a fresh frozen plasma (FFP),

3) The reconstitution solution of claim 1, wherein platelet adhesion and aggregation mediated by the reconstituted plasma is about the same as or greater, as compared to that of the starting plasma prior to spray drying.

4) The reconstitution solution of claim 1, wherein the at least one non-anticoagulant compound that does not bind calcium is selected from the group consisting of glycine HCl, ascorbic acid, lactic acid, gluconic acid and a combination thereof.

5) The reconstitution solution of claim 3, wherein platelet adhesion and aggregation mediated by the reconstituted plasma is measured under arterial shear rate, pathological shear rate, or both.

6) The reconstitution solution of claim 5, wherein under the arterial shear, rate of platelet accumulation is at least about 1% to about 4 times greater than the rate of platelet accumulation of the starting plasma.

7) reconstitution solution of claim 1, wherein platelet adhesion is measured by a flow cell assay.

8) A method of reconstituting spray dried plasma, comprising the steps of:
a) combining a reconstitution solution with spray dried plasma, wherein the spray dried plasma is dried from starting plasma obtained from a donor, wherein the reconstitution solution comprises:
i) a non-anticoagulant compound that does not bind calcium in the range between about 5 mM and about 20 mM;
ii) water;
to obtain reconstituted plasma.

9) The method of claim 8, wherein the method further comprises mixing or shaking the reconstituted plasma.

10) The method of claim 8, wherein step a) occurs in a plasma bag or container.

11) The method of claim 8, wherein the at least one non-anticoagulant compound that does not bind calcium is selected from the group consisting of glycine HCl, ascorbic acid, lactic acid, gluconic acid and a combination thereof.

12) The method of claim 8, wherein a unit of spray dried plasma can be reconstituted with the reconstitution solution at a volume ranging between about 30% and 100% of the starting plasma.

13) A plasma bag or container comprising:
a) a first reconstitution container for storing a reconstitution solution comprising:
i) a non-anticoagulant compound that does not bind calcium in the range between about 5 mM and about 20 mM;
ii) water;
b) a second plasma container for storing spray dry plasma;
c) a connector that communicates between the first container and the second container, the connector having a barrier that can be broken to allow the reconstitution solution to mix with the plasma in the second plasma container.

14) The plasma bag or container of claim 13, wherein the at least one non-anticoagulant compound that does not bind calcium is selected from the group consisting of glycine HCl, ascorbic acid, lactic acid, gluconic acid and a combination thereof.

15) Reconstituted plasma, wherein the reconstituted plasma obtained by mixing spray dried plasma and a reconstitution solution, and wherein the spray dried plasma is dried from a starting plasma that is a never-frozen plasma or thawed from a FFP, reconstitution solution comprises:
i) a non-anticoagulant compound that does not bind calcium in the range between about 5 mM and about 20 mM; and
ii) water;
wherein platelet aggregation and adhesion mediated by the reconstituted plasma is about the same as or greater, as compared to that of the starting plasma prior to spray drying.

16) The reconstitution plasma of claim 15, wherein the at least one non-anticoagulant compound that does not bind calcium is selected from the group consisting of glycine HCl, ascorbic acid, lactic acid, gluconic acid and a combination thereof.

17) The reconstitution plasma of claim 15, wherein platelet adhesion and aggregation mediated by the reconstituted plasma using this reconstitution solution is measured under arterial shear rate, pathological shear rate, or both.

18) The reconstitution plasma of claim 17, wherein under the arterial shear, rate of platelet accumulation is at least about 1% to about 4 times greater than the rate of platelet accumulation of the starting plasma.

19) The reconstitution plasma of claim 15, wherein platelet adhesion is measured by a flow cell assay.

20) Reconstituted plasma comprising:
a) spray dried plasma, wherein the spray dried plasma is dried from a starting plasma that is a never-frozen plasma or thawed from a FFP; and
b) a reconstitution solution comprising:
i) an anticoagulant compound that does not bind calcium in the range between about 5 mM and about 20 mM; and
ii) water;
wherein platelet aggregation and adhesion mediated by the reconstituted plasma is about the same as or greater, as compared to that of the starting plasma prior to spray drying.
